(19) United States
(12) Patent Application Publication
(10) Pub. No.: US 2007/0123768 A1
(43) Pub. Date: May 31, 2007

(54) Ophthalmic instruments, systems and methods especially adapted for conducting simultaneous tonometry and pachymetry measurements

(75) Inventors: Sharon F. Freedman, Durham, NC (US); Phillip H. McKinley, Durham, NC (US); Pratap Challa, Durham, NC (US); Leon W. Herndon, Durham, NC (US); Cynthia A. Toth, Durham, NC (US); Ronald F. Overaker, Durham, NC (US); Brian C. Dodge, Durham, NC (US); Brian Applegate, Durham, NC (US); Joseph Izatt, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY

(21) Appl. No.: 11/289,639

(22) Filed: Nov. 30, 2005

(51) Int. Cl. A61B 3/16; A61B 13/00

(52) U.S. Cl. 600/405; 600/558

(57) ABSTRACT

Ophthalmic instruments, systems and methods enable tonometry and pachymetry measurements to be conducted simultaneously. As such, a patient's intraocular pressure (IOP) corrected for corneal thickness may be determined accurately by a single corneal applanation. Preferably, the ophthalmic instruments have a reference surface at a distal end of the instrument, an applanation plate spaced from the reference surface, and a compliant mount for mounting the applanation plate for resilient displacements relative to the reference surface. The instrument most preferably has a housing such that the reference surface is located at a fixed position at one end of the housing, and the applanation plate is mounted to that one end of the housing in coaxial spaced alignment relative to the reference surface by means of the compliant mount. If desired, the housing may have a handle and a substantially orthogonal head piece containing suitable optic mirrors which allow visible light to pass therethrough so that the procedure can be viewed and/or photographed.

### FIELD OF THE INVENTION

The present invention relates generally to ophthalmic instruments, systems and methods. More specifically, the present invention relates to instruments, systems and methods by which tonometry and pachymetry measurements are conducted.

### BACKGROUND OF THE INVENTION

Goldmann applanation tonometry is a well known technique in the ophthalmic field to measure a patient's intraocular pressure (IOP) which is used as a diagnostic tool for determining eye disorders, such as glaucoma. However, conventional applanation tonometry assumes that each patient has a standard central corneal thickness. Since corneal thickness has a direct impact on the tonometry measurement, the assumption of a standardized corneal thickness means that conventional applanation tonometry can only at best provide a close approximation of the actual IOP from one patient to another.

Goldmann and Schmidt noted the probable relationship of central corneal thickness (CCT) to intraocular pressure (IOP) measurement when they introduced the applanation tonometer in 1956. Multiple reports have confirmed the correlation between increased CCT and increased measured IOP in adults, and a single published report notes a similar finding in children. The Ocular Hypertension Treatment Study highlighted decreased CCT as a predictive factor for progression to glaucoma and other studies have supported the importance of CCT as a contributor to the risk of glaucomatous progression. In addition, black adults demonstrate thinner corneas than whites, and also show increased risk of visual loss from glaucoma.

Both under-diagnosis and over-diagnosis of glaucoma can occur as a result of false intraocular pressure readings. Patients with thinner corneas may register normal intraocular pressures when in fact the pressure within the eye may be high, causing damage to the optic nerve. The reverse also is true in that patients with corneas thicker than normal may have high intraocular pressure readings, yet have normal intraocular pressure and still be treated inappropriately for glaucoma. Accurate and reproducible intraocular pressure readings are not only important for diagnosis of glaucoma but for monitoring the effects of drugs, and laser or incisional surgery. The inability of tonometry alone to detect accurately the true intraocular pressure hampers both detection and treatment. The need for accurate IOP measurements regardless of corneal thickness has also become more important in recent years with the popular practice of surgically altering a patient's cornea for purpose of vision correction. More specifically, patients who have undergone photorefractive keratotomy (PRK) or laser in situ keratomileusis (LASIK) procedures have thinner corneas. These surgically thinned corneas may therefore produce inaccurate and/or misleading IOP readings using current methods, which could lead to an inability to detect glaucoma and loss of sight.

In addition, and perhaps of even more clinical significance is the recent clinical data suggesting that central corneal thickness may be an independent risk factor for glaucomatous damage, even when the IOP has been ‘corrected’ for the measured central corneal thickness. Hence those eyes with thinner than average corneas, seem to be at increased risk for optic nerve damage compared to those with thicker corneas.

For the reasons noted above, it has recently been accepted practice in the ophthalmic field to determine separately the corneal thickness of a patient (e.g., using an ophthalmic pachymeter such as disclosed in U.S. Pat. No. 5,512,966 to Snook¹) in conjunction with conventional applanation tonometry (e.g., using an applanation tonometer such as disclosed in U.S. Pat. Nos. 5,355,884 to Bennett and 4,987,899 to Brown). The tonometer reading is then corrected based on the measured corneal thickness using conventional correction algorithms so as to arrive at a more accurate IOP determination. It has also been proposed in U.S. Pat. No. 6,113,542 to Hyman et al to combine a conventional applanation tonometer with an optical pachymeter having respective separate tonometer and pachymeter probes in a single instrument by which the tonometer signal may be modified using correction algorithms stored in a microprocessor based on the corneal thickness determined previously by the optical pachymeter. A device has also been proposed in U.S. Pat. No. 6,083,161 to O'Donnell, Jr. whereby applanation is done with an ultrasonic transducer which measures the corneal thickness at an exact point of applanation to thereby allow for the simultaneous determination of both applanation pressure and corneal thickness.
¹ All cited patents and written publications are expressly incorporated fully hereinto by reference.

While the techniques employed presently in the art may be satisfactory to improve the accuracy of IOP measurements, there still exists a need for improvement. For example, it would be highly desirable if instruments and methods could be provided which enable ophthalmic tonometry and pachymetry to be conducted simultaneously using a common optical signal path. The importance of a single instrument capable of measuring simultaneously both IOP and central corneal thickness goes beyond convenience. Currently, both of these measurements require that something touch the surface of the anesthetized cornea. While this is an inconvenience to cooperative patients, there are many children and some adults who would greatly benefit by having only one instrumentation of their corneas. Each time an instrument touches the cornea, there is a small disturbance of the surface corneal epithelium, and a topical anesthetic must be placed, which lasts only a few minutes, and which itself can interfere with the blinking response and cause irregularity and even damage to the corneal surface in vulnerable subjects. Therefore, obtaining all needed measurement (i.e., the IOP and central corneal thickness) by a single ‘touch’ to the cornea, poses significant benefit for the patient as well as the eye care provider.

Also, the current algorithms are empirically derived from the average characteristics of a group of test subjects, and therefore result in only an approximation of the IOP correction needed for varying corneal thickness. A more desirable instrument would separate the influence of each cornea on each IOP measurement by an analytic algorithm.

It is towards fulfilling such needs that the present invention is directed.

### SUMMARY OF THE INVENTION

Broadly, the present invention is embodied in ophthalmic instruments, systems and methods which enable contact tonometry and optical pachymetry measurements to be conducted simultaneously. According to the present invention, therefore, ophthalmic instruments, systems and methods are provided whereby a patient's intraocular pressure (IOP) and corneal thickness may be determined accurately by a single path optical signal.

In especially preferred forms, the present invention is embodied in ophthalmic instruments whereby contact tonometry and optical pachymetry measurements are obtained simultaneously (i.e., both measurements are obtained only during a single corneal applanation). Preferably, both the tonometry and pachymetry measurements are obtained optically using a common optical signal path. Alternatively, a tonometer tip associated with a conventional Goldmann contact tonometer employing a pressure sensor to determine applanation pressure may be modified to include optical pachymetry according to the present invention.

According to one embodiment of the present invention ophthalmic instruments are provided which include an applanation tonometer for determining an intraocular pressure measurement, and an optical pachymeter for determining a corneal thickness measurement. The optical pachymeter and tonometer are integral so as to simultaneously generate respective output signals indicative of the intraocular pressure and corneal thickness measurements in response to a single corneal applanation.

In other preferred embodiments of the present invention, both the tonometry and pachymetry measurements are obtained optically using a common optical signal path. Such ophthalmic instruments of the present invention will therefore most preferably include a reference surface at a distal end of the instrument, an applanation plate spaced from the reference surface, and a compliant mount for mounting the applanation plate for resilient displacements relative to the reference surface. The instrument most preferably has a housing such that the reference surface is located at a fixed position at one end of the housing, and the applanation plate is mounted to that one end of the housing in coaxial spaced alignment relative to the reference surface by means of the compliant mount. If desired, the housing may have a handle and a substantially orthogonal head piece containing suitable optic mirrors which allow visible light to pass therethrough so that the procedure can be viewed and/or photographed.

The applanation plate is most preferably circular so that the compliant mount defines an annular mounting region for mounting the applanation plate relative to the reference surface. In this regard, the mounting region is continuous or discontinuous. Preferably, the compliant mount comprises an elastomeric structure, such as an elastomeric O-ring (in which case the annular mounting region is continuous) or a plurality of elastomeric posts circumferentially arranged to define an annular mounting region (in which case the annular mounting region is discontinuous). However, the compliant mount may also be in the form of a plurality of compression springs arranged circumferentially about the applanation plate.

A preferred system for conducting simultaneous tonometry and pachymetry measurements will include an ophthalmic instrument having a reference surface at a distal end of the instrument, an applanation plate spaced from the reference surface; and a compliant mount for mounting the applanation plate for resilient displacements relative to the reference surface, and an interferometer for optical connection to the instrument. The interferometer will most preferably have a source of light to be supplied to the instrument, and a spectrometer for receiving reflected light from the instrument and generating a signal indicative of a patient's intraocular pressure and corneal thickness. A microprocessor is preferably provided to receive the signal generated by the interferometer and determine a patient's intraocular pressure corrected for the patient's corneal thickness.

In use, the ophthalmic instrument of this invention may be brought into alignment with a patient's cornea and advanced toward the cornea so as to cause contact between the cornea and the applanation plate thereof. Continued advancement of the ophthalmic instrument relative to the cornea will cause the applanation plate to be displaced resiliently parallel to and toward the reference surface of the instrument while the cornea is progressively “flattened”. Such advancement of the ophthalmic instrument continues until the cornea is flattened or scanned across this surface sufficiently against the applanation plate. A light beam (preferably annular although another shaped beam or multiple beams could be utilized) may thus be directed toward the flattened cornea so that light may be reflected therefrom. A signal may thus be generated from the reflected light which contains data yielding a patient's intraocular pressure and corneal thickness. In such a manner, therefore, a measurement of the patient's intraocular pressure corrected by the patient's corneal thickness may be obtained.

The measurements and data obtained optically by means of the present invention may also be advantageously used to determine IOP and/or corneal biomechanical properties. For example, the present invention is capable of determining the distance of several points on the cornea from an applanating surface over a range from first corneal contact to full corneal applanation. From such distance data, one can derive factors such as corneal curvature and the applanation area/diameter. Differences of distance to the applanating surface among such points provides a measure of the probes alignment and may thus also be used to drive indicators for the user to assess the quality of the readings. A table with multiple entries of force generated (by the combined effects of IOP and corneal bending) versus applanation diameter can be analyzed to derive an expression of the form F=f1(d)+f2(d), where F is the measured force and d is the applanation diameter. Function f1 is of order 1, and represents the cornea component of force, function f2 is of order 2 and represents IOP component; the internal coefficients required to generate the best fit to the data points in the table indicate the true correction for the force from cornea (which primarily varies with thickness), and the true IOP. Mathematical transformations known to those skilled in the art can be performed with the data from this technology to generate other expressions, e.g., functions of applanation area rather than diameter, and to apply calibration corrections.

These and other aspects and advantages will become more apparent after careful consideration is given to the following detailed description of the preferred exemplary embodiments thereof.

### BRIEF DESCRIPTION OF THE ACCOMPANYING DRAWINGS

Reference will hereinafter be made to the accompanying drawings, wherein like reference numerals throughout the various FIGURES denote like structural elements, and wherein;

FIG. 1 is a schematic cross-sectional view of an ophthalmic instrument which embodies the present invention; lens 22 may be positioned to create a parallel or converging beam, or a focal zone;

FIGS. 2A to 2C are schematic rear perspective views of various embodiments that may be employed to compliantly mount the applanation plate;

FIGS. 3A to 3C are schematic representations of the manner in which the instrument depicted in FIG. 1 may be employed to conduct simultaneous tonometry and pachymetry in accordance with the present invention;

FIGS. 4 and 5 are representative exemplary interferogram plots of optical signal amplitude versus distance from the reference surface for the instrument conditions represented by FIGS. 3B and 3C, respectively;

FIG. 6 is a schematic cross-sectional view of a hand-held ophthalmic instrument which embodies the present invention and allows for visual inspection of the patient's eye; and

FIG. 7 is a schematic representation of another embodiment of the present invention showing a modified applanation tip of a contact tonometer in schematic cross-section which is modified so as permit optical pachymetry measurements to be obtain simultaneously with contact tonometry measurements.

### DETAILED DESCRIPTION OF THE INVENTION

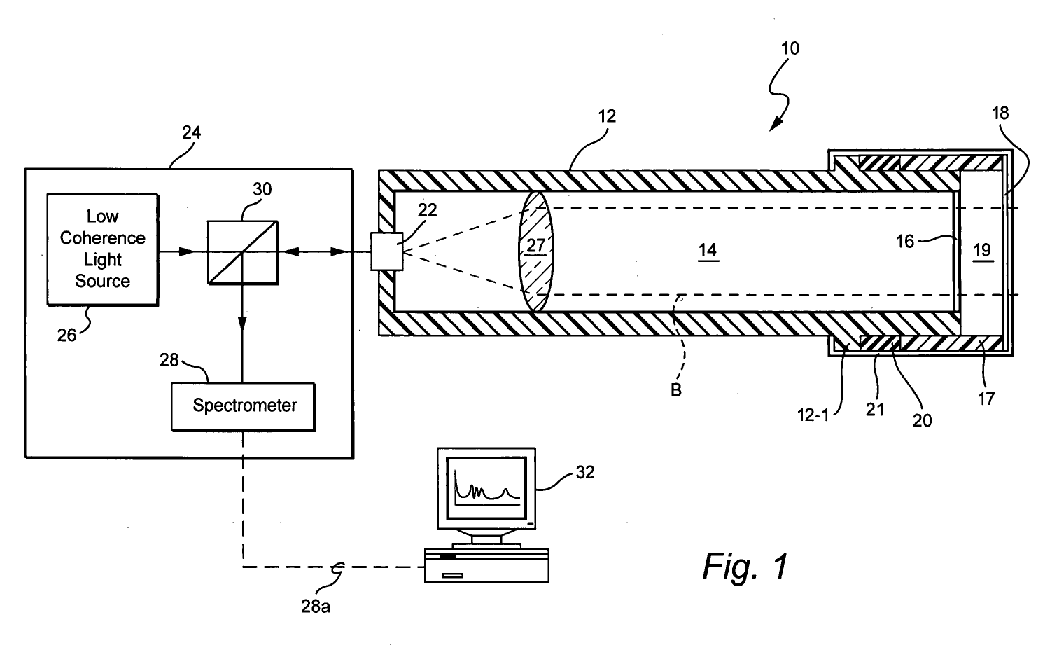

Accompanying FIG. 1 depicts schematically an ophthalmic instrument 10 that embodies one particularly preferred form of the present invention. In this regard, the instrument 10 is most preferably in the form of a hand-held device having a housing 12 defining an interior space 14 terminating in an optically transparent partially reflecting reference surface 16. A cylindrical mounting sleeve 17 is slidably fitted over a distal extremity of the housing 12 and carries at a forward end an optically transparent rigid applanation plate 18. The mounting sleeve 17 thus mounts the applanation plate 18 in coaxially spaced relation to the reference surface 16. Thus, the reference surface 16 is posteriorly spaced from the applanation plate 18 so as to define a cavity space 19 therebetween.

The housing 12 includes an annular stop 12-1 located proximally of the cylindrical mounting sleeve 17. A compliant annular mount 20 is thereby positioned between the mounting sleeve 17 and the stop 12-1 to allow the applanation plate 18 to be capable of parallel resilient displacements towards and away from the reference surface 16.

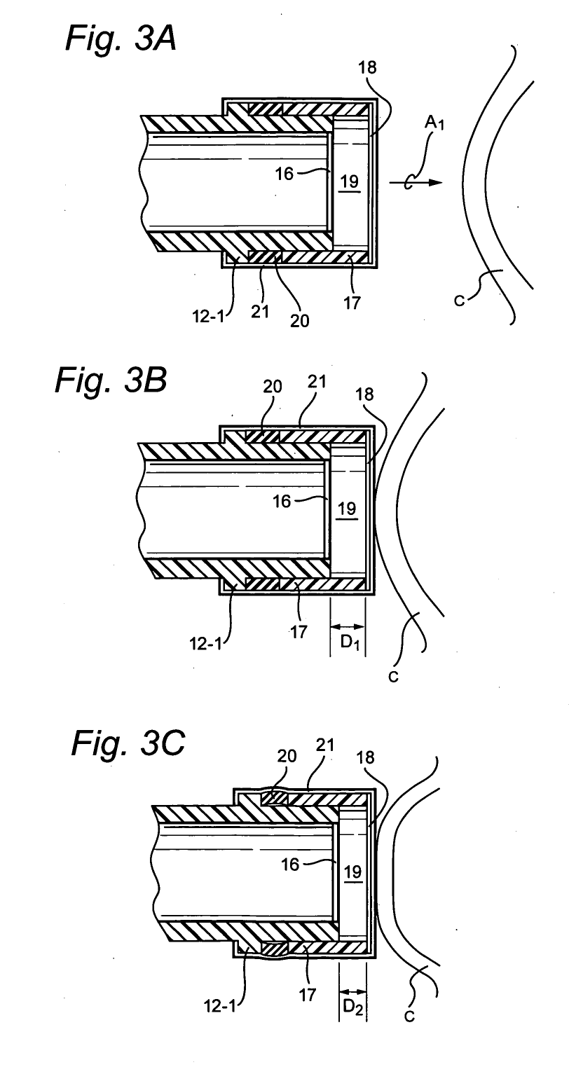

Since the applanation plate 18 is the forwardmost structure of the instrument 10, it is thereby adapted to being brought into physical contact with the surface of a patient's cornea C. (see FIGS. 3A-3C). In order to prevent contamination of the patient's cornea C, it is preferred that a disposable sterile transparent protective cover or “condom” 21 may be provided. The proximal edge of the protective cover 21 may thus be stretched around the stop 12-1 which then serves as the cover's retainer. The protective cover 21 may be made of any suitable transparent material such as polyethylene terephthalate (e.g., MYLAR® film), silicone, polyurethane, polyvinyl chloride (PVC) or the like.

Preferably, the compliant mount 20 is formed of an elastomeric material, such as silicone rubber or the like. Other suitable compliant mounting means may also be envisioned, such as compression springs with mechanical restriction to ensure parallelism in movement of the applanation plate using for example the exterior mounting sleeve 17 depicted in FIG. 1. Alternatively, the mounting sleeve 17 (and hence the annular stop 12-1 and compliant mount 20) could be provided internally of the housing 12. Also, although a cylindrical mounting sleeve 17 has been depicted as being presently preferred, those in this art will recognize that other structurally equivalent mounting assemblies (e.g., multiple piano hinges) could be provided so as to ensure parallelism of the applanation plate 18 and the reference surface 16 during displacements of the former relative to the latter.

The applanation plate 18 is most preferably an optically transparent material that has, or may be made to have, at least about 5% reflectivity at the light wavelengths of the source. Thin films or coatings may thus be provided on the applanation plate 18 and/or reference surface 16 in accordance with well known optical techniques so as to impart desired reflectivity properties. Single crystal sapphire with thickness between 100 and 200 microns is presently preferred as the material from which the applanation plate 18 is constructed though different glasses, acrylics, or other crystals could be used. The applanation plate 18 is also sufficiently rigid so as to remain planar in response to a wide range of intraocular pressure conditions that may be encountered.

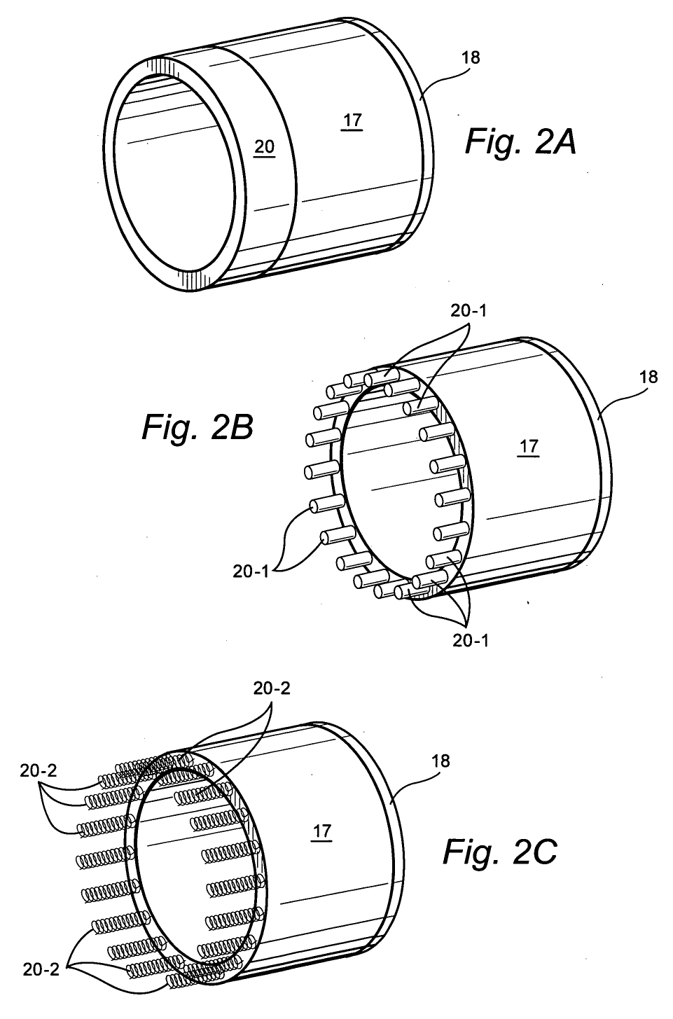

In especially preferred embodiments, the applanation plate 18 has a circular geometry as depicted in FIGS. 2A-2C. The compliant mount 20 therefore in turn most preferably establishes an annular circumferential mounting region relative to the applanation plate 18 which allows the applanation plate 18 to be resiliently displaced substantially uniformly parallel towards and away from the reference surface 16 (i.e., so that the plane of the applanation plate 18 remains substantially parallel to the reference surface 16 throughout its entire range of displacements). Thus, as shown in FIG. 2A the compliant mount 20 may be in the form of a resilient elastomeric O-ring structure and thereby establish a continuous compliant juncture between the applanation plate 18 and the distal end of the housing 12. Alternatively, a discontinuous annular mounting region could likewise be provided by means of structurally individual compliant mounts (e.g., by providing the compliant mount in the form of structurally individual resilient elastomeric post elements 20-1 and/or structurally individual compression springs 20-2 as shown in FIGS. 2B and 2C, respectively). In such a case the number and circumferential spacing of the individual compliant mounts are such that the applanation plate 18 does not skew relative to the reference surface 16 in response to a compression force. The compliant mount 20 (or 20-1, 20-2 and the like) therefore allows substantially uniform planar displacement of the applanation plate 18 from a rest or “zero” position towards the relatively stationary reference surface 16 in response to a compressive force. Once the compressive force is removed, the compliant mount 20 has sufficient resiliency to extend the applanation plate 18 to its rest or “zero” position and thus reestablish the rest or “zero” distance between the applanation plate 18 and the reference surface 16.

Referring again to FIG. 1, it can be seen that the proximal end of the housing 12 is provided with a conventional optical connector 22 for connecting the instrument 10 to an interferometer 24 via a conventional optical signal guide. The interferometer 24 could also be provided as an integral component part of the instrument 10 (e.g., by being integral within the housing 12). The interferometer 24 most preferably comprises a low coherence light source 26 and a spectrometer 28 each being optically connected to a beam splitter 30. The light source 26 serves to provide low coherence light to the instrument 10 whereas the spectrometer receives back scattered light from the instrument 10. The low coherent light source is formed into a circular beam B by internal lens 27 located within the interior space 14 between the proximal and distal ends of the housing 12. Most preferably, the diameter of beam B is substantially 3.06 mm or larger so as to allow measurements equivalent to conventional Goldmann-type tonometer readings to be taken, though with scanning or multiple beams, the diameters would be smaller.

The output signal 28a from the spectrometer 28 is connected to a microprocessor 32 (e.g., a personal computer) by presently preferred means of a conventional USB connection (not shown). Microprocessor 32 stores the algorithms for converting the interferograms data provided by the spectrometer signal 28a into a corrected IOP measurement. Most preferably, the interferometer 24 embodies the principles disclosed more completely in Izatt et al, “Novel Noncontact Optical Pachymeter”, SPIE Ophthalmic Technologies XV Conference, Photonics West, Jan. 22-23, 2005 and Fercher et al, “Measurement of Intraocular Distances by Backscattering Spectral Interferometry”, Optics Communications 117, 43-48 (1995).

Accompanying FIGS. 3A through 3C depict schematically the manner by which simultaneous tonometry and pachymetry measurements may be accomplished. Specifically, FIG. 3A depicts the instrument 10 positioned adjacent to, but spaced from, the patient's cornea C at the beginning of the measurement procedure. The instrument is then advanced toward the cornea C (arrow A₁) until the applanation plate 18 contacts the cornea C as depicted in FIG. 3B. At this point in the procedure, the patient's cornea begins to flatten under pressure of the applanation plate 18. Further advancement of the instrument toward the patient's cornea causes further corneal flattening until the flattened cornea occupies the entire area of the circular light beam from the instrument 10 as depicted in FIG. 3C. Due to the compliant mount 18, the distance between the reference surface 16 and the applanation plate 18 decreases due to intraocular pressure of the patient's eye exerted on the cornea C. Thus, whereas the distance between the reference surface 16 and the applanation plate 18 decreases from distance D₁ upon initial contact with the cornea C as depicted in FIG. 3B to distance D₂ when the cornea C has been completely flattened as depicted in FIG. 3C.

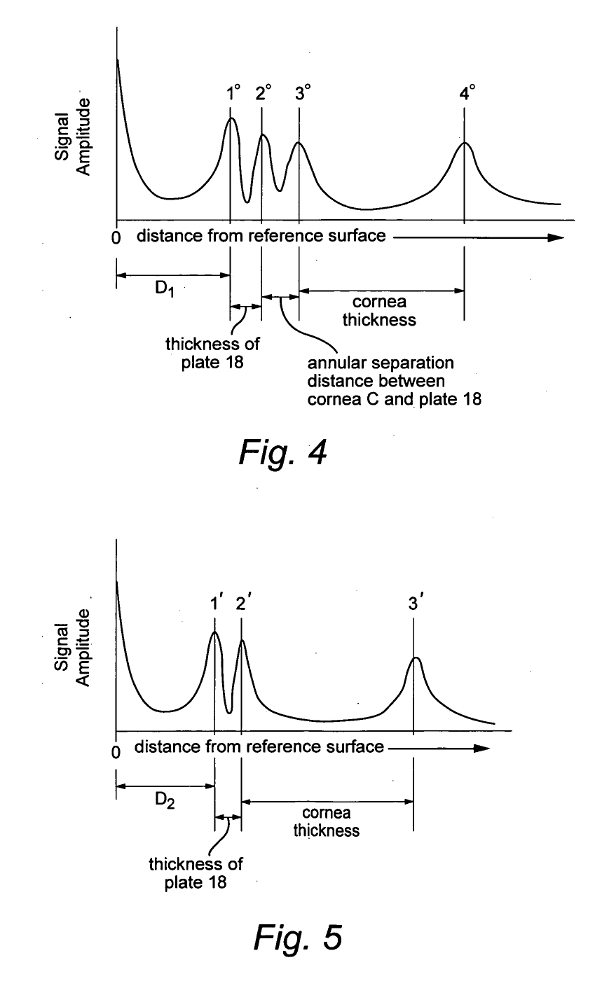

The interferograms generated by the spectrometer 28 corresponding to the instrument conditions depicted in FIGS. 3B and 3C are shown in FIGS. 4 and 5, respectively. In this regard, it will be observed in FIG. 4 that, upon initial contact with the cornea C, the interferogram includes a total of four peaks labeled peaks 1⁰ through 4⁰ from the zero distance position of the reference surface 16. Specifically, the distance from the zero position to peak 1⁰ represents the distance D₁ between the reference surface 16 and the applanation plate 18. The distance between peaks 1⁰ to 2⁰ represents the thickness of the applanation plate 18, while the distance between peaks 2⁰ and 3⁰ represents the annular separation distance between the applanation plate 18 and the cornea C as measured around the central corneal contact point with the applanation plate 18 (i.e., as measured in a region of the diameter of the beam B). Finally, the distance between peaks 3⁰ and 4⁰ represents the thickness of the cornea C.

As the cornea C is flattened, the distance between peaks 2⁰ and 3⁰ trends toward zero when there is no longer any annular separation distance between the applanation plate 18 and cornea C. As shown in FIG. 5, peaks 2⁰ and 3⁰ depicted in FIG. 4 merge into peak 2¹ so that the corneal thickness is measured between peaks 2¹ and 3¹. The area of the flattened cornea achieved by the instrument condition in FIG. 3C will be known since it will is defined by the diameter of the beam B. At zero annular separation distance between the cornea and the applanation plate 18, therefore, the difference between distances D₁ and D₂ will be indicative of the pressure force needed to displace the applanation plate 18 toward the reference surface 16.

It will be appreciated that the actual distance that the applanation plate 18 is displaced from its normal or rest condition will depend on the particular form and material of the compliant mount 20 which can vary from mount to mount or even with the particular instrument's temperature or age. Thus, for any compliant mount 20, the actual displacement distance that the applanation plate 18 moves towards the reference surface 16 will be a function of the magnitude of compression force that is exerted against the applanation plate 18 which those skilled in this art may determine empirically by standard calibration testing before each use. Thus, following such empirical determination of the relationship of the displacement distance and the pressure force for a given compliant mount configuration and/or material, the microprocessor 32 may be provided with an algorithm or look-up table. The displacement distance of the applanation plate 18 relative to the reference surface 16 which is determined by the spectrometer 28 may therefore be converted into a pressure force against the applanation plate 18. This pressure force against the applanation plate 18 will thus correspond to a patient's intraocular pressure condition uncorrected by corneal thickness-that is, an IOP measurement that corresponds to conventional Goldmann-type applanation tonometers. It may be useful to employ an annular beam which is larger than the desired applanation diameter, in which case, peaks 2⁰ and 3⁰ do not merge to peak 2¹, but remain separate (FIGS. 4 and 5). The separation of peaks 2⁰ and 3⁰ is then fixed at a distance that would correspond to having the appropriate applanation diameter, given an average curvature of the cornea. The corneal thickness would then be the distance between peak 2⁰ and 4⁰ (FIG. 4). Having simultaneously determined the corneal thickness in the manner described previously, the microprocessor 32 may thus output an IOP measurement corrected for such corneal thickness using conventional correction algorithms well known to those skilled in this art. The data could also be used to determine corneal biomechanics and corneal curvature.

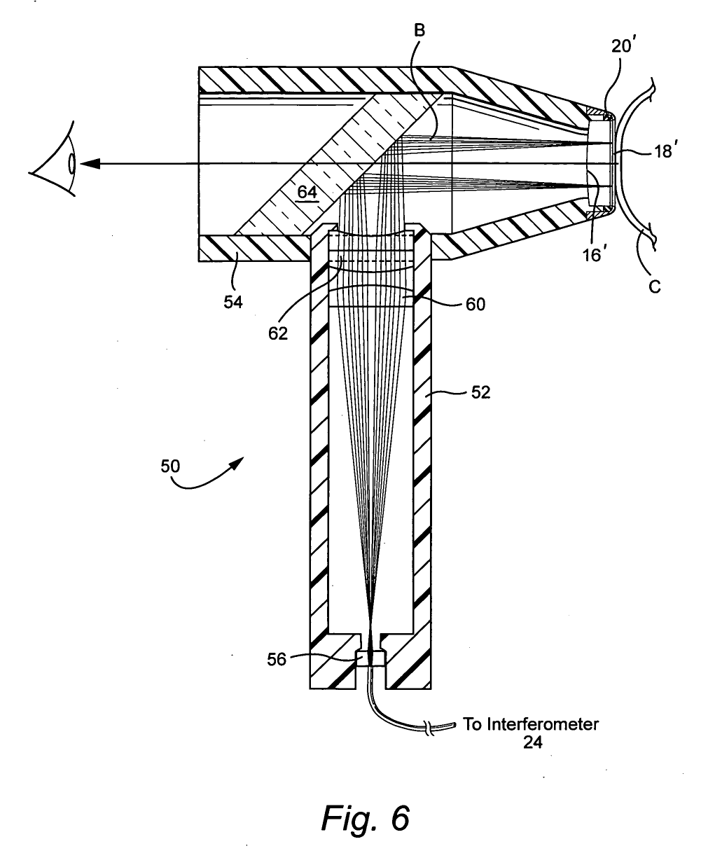

Accompanying FIG. 6 depicts another preferred hand-held instrument 50 which embodies the present invention. Specifically, the instrument 50 includes a handle 52 positioned at an essentially right angle to a headpiece 54. An optical connector 56 is provided at the lower end of the handle 52 so as to connect with interferometer 24 (see FIG. 1). The low coherent light is formed into a circular beam B by means of lenses 60, 62 positioned within the handle 52 which is thereafter redirected by means of a wavelength selective mirror 64. The distal end of the headpiece 54 includes a reference surface 16′, an applanation plate 18′ and an annular compliant mount 20′ which are structurally and functionally similar to the reference surface 16, applanation plate 18 and mount 20 described previously with respect to the embodiment of FIG. 1. The reflected light of visible wavelengths is thus allowed to pass through the mirror 64 to allow viewing and/or video or photographic recording.

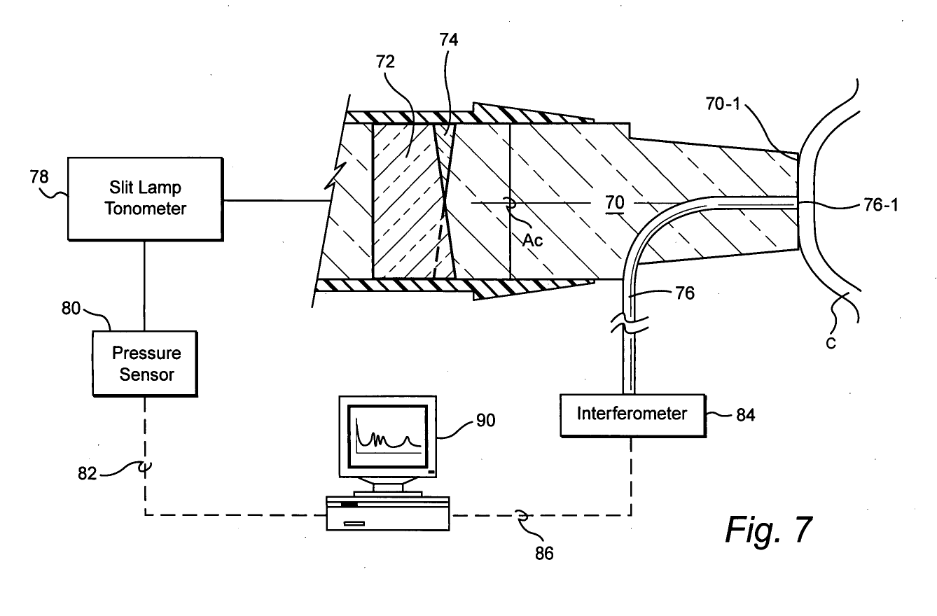

The embodiments described above employ optical means for simultaneously obtaining both tonometry and pachymetry measurements. However, according to the present invention conventional tonometers could be modified so that optical pachymetry measurements could be obtained simultaneously with conventional tonometry measurements using standard electromechanical pressure sensors. Such an embodiment of the present invention is depicted in accompanying FIG. 7 whereby a tonometer tip 70 having internal prisms 72, 74 includes an optical fiber 76 embedded within the tip 70. The optical fiber 76 includes a terminal end 76-1 which is coplanar with the applanation surface 70-1 of the applanation tip 70 and coaxially disposed relative to the applanation tip's central axis A_{c}. As is in and of itself conventional, the applanation tip 70 is connected operatively to a slit lamp tonometer 78 that may be operated manually by the attending professional using techniques will known to those in the optometry art. A pressure sensor 80 is operatively coupled to the tonometer 78 so as to sense the IOP reading obtained by the tonometer 78 during applanation of the patient's cornea C. The pressure sensor 80 outputs a signal via line 82 indicative of the IOP measurement obtained by the tonometer 78.

The optical fiber 76 is coupled operatively to an interferometer 84 having characteristics similar to the interferometer 24 described previously. The interferometer 84 will thus output a signal via line 86 which is indicative of the thickness of the patient's cornea C. Thus, simultaneously with corneal applanation by the applanation surface 70-1 and the generation of the tonometry signal 82, the interferometer 84 will output a pachymetry signal via line 86. These simultaneously generated tonometry and pachymetry signals 82, 86, respectively, are received by microprocessor 90 (e.g., a personal computer) which converts the data signals into a corrected IOP measurement using algorithms according to the techniques described previously.

It is entirely conceivable that the instruments and systems described fully herein, while being especially suited for the simultaneous measurement of a patient's IOP and corneal thickness, could be employed to determine such measurements separately. Thus, if desired, the instruments and systems described herein could be employed so as to determine separately one of the IOP and corneal thickness if that were deemed desirable. Thus, the microprocessor could be configured to provide a readout of the patient's IOP (e.g., as determined by the displacement distance of the applanation plate 18, in which case the IOP measurement would be commensurate with conventional Goldmann-type tonometer readings at a full applanation diameter of 3.06 mm) and/or a readout of the patient's corneal thickness. In other words, while it may be very desirable to conduct simultaneous measurements of both IOP and corneal thickness, the instruments and systems are sufficiently flexible to permit separate measurement determinations if desired.

Therefore, while the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. An ophthalmic instrument comprising:
instrument means for determining simultaneously an intraocular pressure measurement in response to corneal applanation, and a corneal thickness measurement in response to corneal reflection of light during corneal applanation; and
signal generating means for generating signals indicative of the simultaneously determined intraocular pressure and corneal thickness measurements.

2. An ophthalmic instrument as in claim 1, wherein the instrument means comprises an applanation tonometer having an applanation tip defining an application surface, a slit lamp tonometer operatively connected to the applanation tip, and a pressure sensor operatively connected to the slit lamp tonometer for outputting the signal indicative of intraocular pressure measurement.

3. An ophthalmic instrument as in claim 2, wherein the instrument means comprises a pachymeter having an optical fiber with a terminal end which is coaxially embedded within the applanation tip so as to be coplanar with the applanation surface, and an interferometer operatively connected to the optical fiber for receiving corneal reflection of light and outputting the signal indicative of corneal thickness measurement based thereon.

4. An ophthalmic instrument as in claim 1, wherein the instrument means comprises:
an applanation surface for contact with a cornea; and
an interferometer system for determining simultaneously intraocular pressure and corneal thickness conditions in response to optical signals propagating along a common optical signal path between the applanation surface and the interferometer.

5. An ophthalmic instrument as in claim 1, wherein the instrument means comprises:
an applanation tonometer for determining an intraocular pressure measurement; and
an optical pachymeter for determining a corneal thickness measurement; wherein
the tonometer and optical pachymeter are integral so as to simultaneously generate respective output signals indicative of the intraocular pressure and corneal thickness measurements in response to a single corneal applanation.

6. An ophthalmic instrument as in claim 1, wherein the instrument means comprises:
(i) a reference surface;
(ii) an applanation plate spaced from the reference surface; and
(iii) a compliant mount for mounting the applanation plate for resilient displacements relative to the reference surface.

7. An ophthalmic instrument comprising:
(i) a reference surface at a distal end of the instrument;
(ii) an applanation plate spaced from the reference surface; and
(iii) a compliant mount for mounting the applanation plate for resilient displacements relative to the reference surface.

8. The instrument of claim 7, further comprising a housing, wherein
the reference surface is located at a fixed position at one end of the housing, and wherein
the applanation plate is mounted to the one end of the housing in coaxial spaced alignment relative to the reference surface by the compliant mount

9. The instrument of claim 8, further comprising an optical connector at another end of the housing for receiving an optical signal guide.

10. The instrument of claim 8, wherein the housing comprises a handle and a headpiece connected to the handle, wherein the reference surface is located at a fixed position at a distal end of the headpiece.

11. The instrument of claim 10, further comprising an optical connector at a lower end of the handle.

12. The instrument of claim 10, wherein the headpiece comprises a proximal end for viewing, and wherein the instrument comprises a wavelength selective mirror for allowing reflected light of a visible wavelength to be viewed at the proximal end of the headpiece and for allowing light of other wavelengths to be reflected between the distal end of the headpiece and the lower end of the handle.

13. The instrument of claim 10, further comprising a lens for forming light into a circular light beam.

14. The instrument of claim 7, wherein the applanation plate is circular.

15. The instrument of claim 7, wherein the compliant mount defines an annular mounting region for mounting the applanation plate relative to the reference surface.

16. The instrument of claim 15, wherein the mounting region is continuous or discontinuous.

17. The instrument of claim 7, wherein the compliant mount comprises an elastomeric structure.

18. The instrument of claim 17, wherein the elastomeric structure comprises an elastomeric O-ring.

19. The instrument of claim 17, wherein the elastomeric structure comprises a plurality of elastomeric posts circumferentially arranged to define an annular mounting region.

20. The instrument of claim 7, wherein the compliant mount comprises a compression spring.

21. The instrument of claim 20, wherein the compliant mount comprises a plurality of compression springs circumferentially arranged to define an annular mounting region.

22. A ophthalmic system for conducting simultaneous ocular tonometry and pachymetry measurements comprising:
an instrument as in claim 7; and
an interferometer system operatively connected to the instrument for determining simultaneously intraocular pressure and corneal thickness conditions in response to optical signals propagating along a common optical signal path between the applanation plate and the interferometer.

23. A ophthalmic system for conducting simultaneous ocular tonometry and pachymetry measurements comprising:
an instrument having an applanation plate for contact with a cornea; and
an interferometer system operatively connected to the instrument for determining simultaneously intraocular pressure and corneal thickness conditions in response to optical signals propagating along a common optical signal path between the applanation plate and the interferometer.

24. The ophthalmic system of claim 23, further comprising a microprocessor operatively connected to the interferometer system.

25. The ophthalmic system of claim 23, wherein the instrument comprises a compliant mount which mounts the applanation plate for compliant displacements in response to corneal contact.

26. The ophthalmic system of claim 25, wherein the interferometer system optically determines intraocular pressure conditions in response to compliant displacement of the applanation plate.

27. The ophthalmic system of claim 26, wherein the interferometer system comprises (a) a source of light to be supplied to the instrument along the common optical signal path, and (b) a spectrometer for receiving reflected light from the instrument along the common optical signal path and generating a signal indicative of the intraocular pressure and corneal thickness.

28. The ophthalmic system of claim 25, wherein the compliant mount comprises an elastomeric or spring member.

29. The ophthalmic system of claim 28, wherein the compliant mount comprises a plurality of individual elastomeric or spring members.

30. A system for conducting simultaneous ocular tonometry and pachymetry measurements comprising:
an ophthalmic instrument having (i) a reference surface at a distal end of the instrument; (ii) an applanation plate spaced from the reference surface; and (iii) a compliant mount for mounting the applanation plate for resilient displacements relative to the reference surface; and
an interferometer for optical connection to the instrument, wherein
the interferometer comprises (a) a source of light to be supplied to the instrument, and (b) a spectrometer for receiving reflected light from the instrument and generating a signal indicative of a patient's intraocular pressure and corneal thickness.

31. The system of claim 30, further comprising a microprocessor for receiving the signal generated by the interferometer and determining a patient's intraocular pressure corrected for the patient's corneal thickness.

32. A method for conducting simultaneous tonometry and pachymetry measurements of a patient's eye, comprising:
(a) applanating a patient's cornea; and
(b) simultaneously while applanating the patient's cornea according to step (a), determining an intraocular pressure measurement in response to pressure of the applanated cornea and a corneal thickness measurement in response to optical illumination of the applanated cornea.

33. The method of claim 32, wherein step (a) comprises:
(a1) bringing into alignment with a patient's cornea an ophthalmic instrument having (i) a reference surface at a distal end of the instrument; (ii) an applanation plate spaced from the reference surface; and (iii) a compliant mount for mounting the applanation plate for resilient displacements relative to the reference surface;
(a2) advancing the ophthalmic instrument toward the cornea so as to cause contact between the cornea and the applanation plate thereof; and
(a3) continuing to advance the ophthalmic instrument relative to the cornea to cause the applanation plate to be displaced resiliently toward the reference surface of the instrument until the cornea is flattened sufficiently against the applanation plate; and
wherein step (b) comprises:
(b1) directing a circular beam of light toward the flattened cornea and allowing light to be reflected therefrom; and
(b2) generating a signal from the reflected light which is indicative of a patient's intraocular pressure and corneal thickness.

34. A method for conducting simultaneous tonometry and pachymetry measurements of a patient's eye, comprising:
(a) bringing into alignment with a patient's cornea an ophthalmic instrument having (i) a reference surface at a distal end of the instrument; (ii) an applanation plate spaced from the reference surface; and (iii) a compliant mount for mounting the applanation plate for resilient displacements relative to the reference surface;
(b) advancing the ophthalmic instrument toward the cornea so as to cause contact between the cornea and the applanation plate thereof;
(c) continuing to advance the ophthalmic instrument relative to the cornea to cause the applanation plate to be displaced resiliently toward the reference surface of the instrument until the cornea is flattened sufficiently against the applanation plate; and
(d) directing a circular beam of light toward the flattened cornea and allowing light to be reflected therefrom; and
(e) generating a signal from the reflected light which is indicative of a patient's intraocular pressure and corneal thickness.

35. The method of claim 34, wherein step (e) is practiced to obtain a measurement of the patient's intraocular pressure corrected by the patient's corneal thickness.

36. The method of claim 35, wherein step (e) is practiced using a microprocessor.

37. An ophthalmic instrument system comprising:
an ophthalmic instrument having an applanation plate adapted to be placed adjacent to a patient's cornea, a reference surface posteriorly spaced from the applanation plate, and compliant mounting means for mounting the applanation plate for resilient displacements relative to the reference surface;
a light source for providing light to the ophthalmic instrument; and
spectrometer means for receiving reflected light from the ophthalmic instrument and determining therefrom a displacement distance by which the applanation plate is moved resiliently towards the reference surface in response to a pressure force applied against the applanation plate by the patient's cornea in contact therewith and generating a first distance signal indicative of the displacement distance; and
a microprocessor for receiving the first distance signal from the interferometer means and determining therefrom a patient's intraocular pressure.

38. The ophthalmic instrument system as in claim 37, wherein the spectrometer means receives reflected light from the patient's cornea and generates a second distance signal which is indicative of a corneal thickness dimension, and wherein the microprocessor receives the second distance signal to determine therefrom the patient's corneal thickness dimension.

39. The ophthalmic instrument system of claim 38, wherein the microprocessor uses the first and second distance signals to determine therefrom the patient's intraocular pressure condition corrected by the patients corneal thickness dimension.
